(19) United States
(12) Patent Application Publication
(10) Pub. No.: US 2012/0053220 A1
(43) Pub. Date: Mar. 1, 2012

(54) NOVEL LIPOXYGENASE INHIBITORS AS NEUROPROTECTIVE AGENTS

(75) Inventors: Klaus Van Leyen, Cambridge, MA (US); Theodore R. Holman, Santa Cruz, CA (US); Eng H. Lo, Newton, MA (US); Matthew P. Jacobson, San Francisco, CA (US); David W. Howells, North Balwyn (AU)

(73) Assignees: The General Hospital Corporation d/b/a Massachusetts General Hospital, Boston, MA (US); Florey Neuroscience Institutes, Heidelberg, Victoria (AU); The Regents of the University of California, Oakland, CA (US)

(21) Appl. No.: 12/671,567

(22) PCT Filed: Aug. 1, 2008
(86) PCT No.: PCT/US2008/009327

§ 371 Date: Oct. 19, 2011

Related U.S. Application Data
(60) Provisional application No. 60/963,104, filed on Aug. 2, 2007.

(51) Int. Cl. A61K 31/415; A61P 25/16; A61P 25/28; C12N 9/99; A61K 31/381

(52) U.S. Cl. 514/407; 435/184; 514/443

(57) ABSTRACT

The present invention relates to the identification of inhibitors of lipoxygenase enzymes. Methods are presented for using novel lipoxygenase inhibitors: LOXBlock-1 and LOXBlock-3, and other candidate lipoxygenase inhibitors identified by similar screening strategies, in therapy and diagnostics for neurodegenerative disorders.

### RELATED APPLICATIONS

This application claims the benefit under 35 U.S.C. §119(e) of U.S. provisional application Ser. No. 60/963,104, filed Aug. 2, 2007, the disclosure of which is incorporated by reference herein in its entirety.

### GOVERNMENT INTEREST

This work was funded in part by the National Institutes of Health under grant numbers NS049430 and GM56062. The government has rights in this invention.

### FIELD OF THE INVENTION

The present invention relates to the identification of lipoxygenase inhibitors and the treatment of neurodegenerative disorders using such inhibitors.

### BACKGROUND OF THE INVENTION

Oxidative stress is a major mechanism implicated in a variety of neurodegenerative diseases including stroke, Alzheimer's disease, and Parkinson's disease (Lin and Beal 2006). 12/15-LOX, also known as leukocyte-type 12-LOX in mice and 15-LOX-1 in humans (Brash 1999; Yamamoto et al. 1999), may be one of the key mediators in neurodegenerative disease, because it is triggered by reactive oxygen species (ROS). Once activated, 12/15-LOX generates lipid hydroperoxides that serve to further amplify oxidative stress (Kuhn et al. 1990). While lipoxygenases typically oxidize free polyunsaturated fatty acids such as arachidonic acid, 12/15-LOX can also directly oxidize and damage organelle membranes (Kuhn et al. 1990; van Leyen et al. 1998). Elevated amounts of 12/15-LOX have been found in experimental stroke in mice (van Leyen et al. 2006), and in early phases of Alzheimer's in humans (Pratico et al. 2004). Cell culture studies have implicated 12/15-LOX in neuronal models of oxidative stress related to Alzheimer's (Lebeau et al. 2004; Zhang et al. 2004) and Parkinson's diseases (Li et al. 1997; Mytilineou et al. 2002). Finally, 12/15-LOX knockout mice are protected in middle cerebral artery occlusion (MCAO) models of stroke (Khanna et al. 2005; van Leyen et al. 2006). All of these studies suggest that finding novel inhibitors of 12/15-LOX may expand treatment options for these neurodegenerative diseases.

At the present time, drug discovery is still a tedious process, with multiple rounds of complicated experiments, both in vitro and in vivo, each of which can lead to failure for any given drug candidate. Any approach that reduces either the number of testing rounds, the complexity of the assays involved, or the number of compounds to be tested in vivo, would constitute substantial progress in drug discovery.

### SUMMARY OF THE INVENTION

One approach that has recently come to the forefront is virtual screening of chemical libraries, based on known protein structures either derived from X-ray crystallographic studies, or computer-generated based on known structures (Jacobson and Sal12004). Compared to random screening of unknown compounds, this approach can increase the likelihood of finding specific inhibitors of a given enzyme, since modeling is based on an interaction of the drug candidate with the active site of the target enzyme. A novel screening method was developed to identify inhibitors of 12/15-LOX. The method involves a virtual computer screen, followed by verification of neuroprotective qualities in cultured neuronal cell lines and primary neuronal and oligodendroglial cells. The invention further discloses methods for using two compounds identified through this screening method, LOXBlock-1 and LOXBlock-3, for treatment of neurodegenerative disorders such as stroke.

Aspects of the invention relate to methods for treating a neurodegenerative disorder in a subject by administering to a subject having or suspected of having a neurodegenerative disorder a pharmaceutical composition comprising LOXBlock-1, LOXBlock-3 and/or any combination thereof in an amount effective to treat the neurodegenerative disorder. In some embodiments the neurodegenerative disorder is stroke. In other embodiments, the neurodegenerative disorder is Alzheimer's disease, Parkinson's disease, or periventricular leukomalacia (PVL).

In some embodiments the subject having the neurodegenerative disorder is a human. The pharmaceutical composition containing LOXBlock-1, LOXBlock-3 and/or any combination thereof may further comprise at least one pharmaceutically acceptable carrier, diluent, excipient or adjuvant. The method of administering the pharmaceutical composition may include parenteral, oral, buccal, pulmonary, intravenous, intramuscular, subcutaneous, aural, rectal, vaginal, ophthalmic, intradermal, intraoccular, intracerebral, intralymphatic, intraarcticular, intrathecal or intraperitoneal.

Aspects of the invention relate to methods for protecting a cell against oxidative-stress-related injury by inhibition of 12/15-LOX activity, through contacting a cell undergoing oxidative-stress-related injury with LOXBlock-1, LOX-Block-3 and/or any combination thereof in an amount effective to inhibit 12/15-LOX activity. In some embodiments the cells that are undergoing oxidative stress are neuronal or oligodendroglial, and may be in vitro or in vivo. The cells at risk of oxidative-stress-related injury may be human or non-human cells, and may comprise more than one cell type. In some embodiments the cells are rodent cells such as HT22 cells. In some embodiments the oxidative-stress-related injury may comprise a neurodegenerative disorder, such as stroke.

Further aspects of the invention relate to a method for identifying compounds useful for treating neurodegenerative disease. The multi-step method comprises: (a) testing virtual compounds for compounds that bind to a three dimensional structure or a homology model of a lipoxygenase protein, (b) selecting compounds that bind to the three dimensional structure or homology model of the lipoxygenase protein and testing them for the ability to inhibit the activity of the lipoxygenase protein in vitro, (c) selecting compounds that have the ability to inhibit the activity of the lipoxygenase protein in vitro and testing them for neuroprotective activity in cells, and (d) selecting compounds that have neuroprotective activity in cells and testing them for neuroprotective activity in primary neurons and/or oligodendrocytes. Compounds found to have neuroprotective activity in primary neurons and/or oligodendrocytes would be considered compounds useful for treating neurodegenerative disease. In some embodiments the lipoxygenase protein used for screening is 12/15-LOX.

In certain embodiments the virtual compounds are tested for their ability to bind to the active site of 12/15-LOX. The test for neuroprotective activity in cells may be performed in human or non-human cells. In some embodiments the test for neuroprotective activity is performed in rodent cells such as HT22 cells. The test for neuroprotective activity in primary neurons and/or oligodendrocytes may be performed in human or non-human primary neurons and/or oligodendrocytes. In some embodiments the test for neuroprotective activity in primary neurons and/or oligodendrocytes is performed in rodent primary neurons and/or oligodendrocytes. In some embodiments, lipoxygenase inhibitor compounds identified through this screening method may be used to treat neurodegenerative disorders such as stroke, Alzheimer's disease, Parkinson's disease, or periventricular leukomalacia (PVL).

Further aspects of the invention relate to methods for imaging a stroke-related infarction in a patient by administering to the patient an effective amount of LOXBlock-1, LOXBLock-3 and/or any combination thereof, and detecting the LOXBlock-1, LOXBLock-3 and/or any combination thereof. In some embodiments LOXBlock-1, LOXBLock-3 and/or any combination thereof is detected by positron emission tomography (PET) or single photon emission computed tomography (SPECT) imaging. In certain embodiments, LOXBlock-1, LOXBLock-3 and/or any combination thereof is associated with a label such as a radionuclide or a paramagnetic contrast agent. The detection of LOXBlock-1, LOXBLock-3 and/or any combination thereof indicates the presence of 12/15-LOX protein.

The foregoing compounds and compositions can be used for manufacturing a medicament including medicaments for the treatment of disorders including neurodegenerative disorders and oxidative-stress-related injury.

These and other aspects of the invention will be described in greater detail below.

### BRIEF DESCRIPTION OF THE DRAWINGS

FIG. 1 depicts the chemical structures of the LOX inhibitors LOXBlock-1, LOXBlock-2 and LOXBlock-3 (A), a graph demonstrating the results of an antioxidant activity assay using LOXBlock-1, LOXBlock-2 and LOXBlock-3 (B), and a graph demonstrating the results of a neuroprotection assay in HT22 cells using LOXBlock-1, LOXBlock-2 and LOXBlock-3 (C).

FIG. 2 presents six graphs demonstrating the results of neuroprotection assays in rat primary neurons (A-C) and in oligodendroglial cells (D-F), using LOXBlock-1, LOXBlock-2 and LOXBlock-3.

### DETAILED DESCRIPTION OF THE INVENTION

The lipid-metabolizing enzyme 12/15-lipoxygenase (12/15-LOX) mediates cell death due to oxidative stress in both neurons and oligodendrocytes. Specifically, it may contribute to the pathophysiology of stroke, Alzheimer's and Parkinson's disease. The invention relates at least in part to the finding that two out of three specific 12/15-LOX inhibitors, derived from a virtual screen by computational modeling and validated by inhibition of recombinant human 15-LOX in vitro, are able to rescue both neuronal as well as oligodendroglial cells from cell death induced by oxidative stress. Thus, in a streamlined process, an initial virtual screen of 50,000 compounds in a library of drug-like molecules led to the identification of two novel drug candidates for targeting 12/15-LOX: LOXBlock-1 and LOXBlock-3.

The neuroprotective abilities of LOXBlock-1 and LOXBlock-3 were unexpected because a third 12/15-LOX inhibitor, LOXBlock-2, derived from the same virtual screen, inhibited 15-LOX in vitro but was not neuroprotective, instead demonstrating cell lethality. Thus the virtual screening assay alone could not predict whether a molecule that binds to 12/15-LOX would also be neuroprotective, emphasizing the importance of follow-up in vivo assays combined with the virtual screening method to identify candidate lipoxygenase inhibitors. In addition, LOXBlock-1 and LOXBlock-3 were unexpectedly found to protect primary brain cells from lipoxygenase-mediated cell death at lower concentrations than were required for inhibition of 15-LOX in vitro, further emphasizing the importance of in vivo assays combined with virtual screening to identify candidate lipoxygenase inhibitors.

The novel inhibitors of 12/15-LOX, LOXBlock-1 and LOXBlock-3 may provide new therapeutic opportunities to combat stroke and other neurodegenerative diseases. Aspects of the invention relate to methods for using LOXBlock-1 and LOXBlock-3 for treatment and diagnosis of neurodegenerative disorders such as stroke, and methods for further identifying LOX inhibitors.

### Lipoxygenase Proteins

Lipoxygenase proteins (also called LOX or LO proteins) are non-heme, iron-containing enzymes that mediate the oxidation of polyunsaturated fatty acids containing a cis-double bond; essential fatty acids in humans. Lipoxygenases are found in a wide variety of plants, fungi and animals. The nomenclature for mammalian lipoxygenases is based on the prototypical tissue of their occurrence and their specificity against arachidonic acid (AA), with the majority oxygenating on either carbon 5, 12 or 15. Lipoxygenase proteins modulate a wide variety of biological processes and have been linked to multiple disease states including cancer, asthma, heart disease and neurodegenerative disorders. Human lipoxygenases 5-hLOX, 12-hLOX and 15-hLOX have all been determined to have some role in uncontrolled cell growth and/or regulation of cancerous tissue.

In addition, 5-, 12-, and 15-LOX are all expressed in the brain (reviewed in Phillis, J. W. et al. (2006) Brain Res. Rev. 52:201-43). The lipoxygenase proteins that have primarily been linked to neurodegenerative disease are members of the 12/15-LOX subfamily (Feinmark, S. J. et al. (2003) J. Neurosci. 23:11427-11435; Pratico, D. et al. (2004) Am. J. Pathol. 164:1655-1662; Chinnici, C. M et al. (2005) Am. J. Pathol. 167:1371-77). 12/15-LOX proteins, which are expressed prominently in the nervous system, are dual specific, meaning that they produce both 12-hydroxyeicosatetraenoic acid (12(S)-HETE) and 15(S)-HETE from arachidonic acid. Human 15-LOX-1 is encoded by the gene ALOX15 (Sigal, E. et al. (1988) Biochem. Biophys. Res. Commun. 157:457-464). A likely mouse ortholog of human 15-LOX-1 is 12(S)-LOX (Schneider, C. et al (2004) J. Invest. Dermatol. 122:691-98). Lipoxygenase proteins are discussed further in US Patent Publications: 20070136832, 20070134680, and 20040137483.

Recent gene array analysis has shown that 12-LOX is up-regulated after hypoxia in neonatal rat brain (Bernaudin, M et al. (2002) J. Biol. Chem. 277: 39728-38) Moreover, in a related model of ischemic injury, 12-LOX was found to be up-regulated 25-fold in rat after spinal cord injury (Di Giovanni, S. et al. (2003) Ann. Neurol. 53:454-68) Finally, mice in which the gene encoding 12-LOX has been deleted, are protected in middle cerebral artery occlusion models of stroke (Khanna et al., 2005; van Leyen et al., 2006). All of these studies suggest that finding novel inhibitors of 12-LOX may be beneficial to expanding treatment options for stroke and other neurodegenerative disorders.

### Lipoxygenase Inhibitors

In order to understand the cellular function of lipoxygenase proteins it is to advantageous to discover potent and specific inhibitors to particular LOX isozymes. Many terrestrial natural products have been discovered over the years that inhibit 15-hLOX-1, such as boswellic acid (IC₅₀=1 uM), hexamethoxyflavone (IC₅₀=50 uM), nor-dihydroguaiaretic acid (NDGA) (IC₅₀=0.5 uM), baicalein (IC₅₀=2 uM) and bacterial hopanoids (IC₅₀=10 uM), but few are known to be specific to either 15-hLOX-1 or 12-hLOX. For many inhibitors, the specificity and inhibition mechanism have never been characterized. In general, there are three broad categories of LOX inhibitors: reductive, catalytic and allosteric. The reductive inhibitors convert the active, ferric enzyme to the inactive, ferrous form. The catalytic inhibitors are standard affinity molecules, such as competitive inhibitors. The allosteric inhibitors, however, have markedly different kinetic properties and bind to a site distinct from the catalytic site. The exact location of the allosteric site is still unknown, however, its presence allows researchers to target two sites in lipoxygenase, the allosteric and catalytic sites, which may have different structure/activity relationships and thus different pharmacophore profiles.

### LOXBlock-1 and LOXBlock-3

Two novel lipoxygenase inhibitors were recently identified through a virtual screening approach using the ChemBridge Diversity set (Kenyon et al., 2006). LOXBlock-1 and LOXBlock-3 were identified as binding to homology models of human platelet-type 12-lipoxygenase (12-hLOX) and human reticulocyte 15-lipoxygenase-1 (15-hLOX). LOXBlock-1 (2-amino-N-(3-chlorophenyl)-4,5,6,7-tetrahydrobenzothiophene-3-carboxamide; ChemBridge ID 5680672) has a molecular mass of 306.81 Da, and can inhibit both 12-hLOX and 15-hLOX with IC₅₀ values of 30.7±6.8 μM and 18.8±4.7 μM respectively. The molecular structure of LOXBlock-1 is shown below (a).

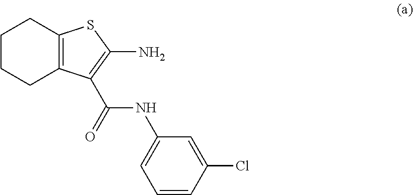

LOX-Block-3 (4-ethyl-6-(4-phenoxy-1H-pyrazol-3-yl)benzene-1,3-diol; ChemBridge ID 6640337) has a molecular mass of 296.32 Da, and can inhibit both 15- and 12-hLOX with IC₅₀ values of 9.2±1.4 μM and 12.3±0.9 μM respectively. The molecular structure of LOX-Block-3 is shown below (b).

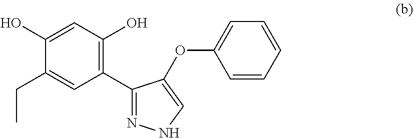

LOXBlock-1 and LOX-Block-3 bind to the catalytic site of 12/15 LOX and inhibit LOX activity in vitro. As shown herein, LOXBlock-1 and LOXBlock-3 exhibit neuroprotective activity in cell-culture, primary neurons, and in oligodendroglial cells, making these compounds promising candidates for therapeutic and diagnostic approaches to targeting neurodegenerative disorders which demonstrate increased lipoxygenase expression or activity. In certain embodiments of the invention, LOXBlock-1 or LOXBlock-3 may be used separately for therapeutic or diagnostic purposes. In other embodiments LOXBlock-1 and LOXBlock-3 may be used together for therapeutic or diagnostic purposes. It should also be appreciated that LOXBlock-1 and/or LOXBlock-3 may be combined with other LOX inhibitors for therapeutic or diagnostic purposes.

### Methods for Identifying Inhibitors

The success of the virtual screening method in identifying LOXBlock-1 and LOXBlock-3 as compounds that are capable of inhibiting lipoxygenase activity and exhibiting neuroprotective activity, indicates the usefulness of this approach, combined with subsequent cell-based screens, in identifying compounds that could potentially be used to treat neurodegenerative disorders.

Aspects of the invention relate to methods for identifying further compounds that may be useful for therapeutic and/or diagnostic approaches to neurodegenerative disorders. An initial step in the screening process involves conducting virtual screens to identify compounds that bind to lipoxygenase enzymes, and are therefore potential inhibitors of lipoxygenase enzymes. In some embodiments the lipoxygenase enzyme is 12-hLOX or 15-hLOX. However it should be appreciated that using the methods described in the instant invention, virtual screening could be conducted to identify compounds that interact with any of the lipoxygenase enzymes from any species. As used herein, conducting a virtual screen refers to conducting a screen that uses models of compounds, rather than conducting a screen with the physical compounds. In some embodiments the three-dimensional structure of a lipoxygenase protein may be empirically determined, for example by X-ray crystallography or NMR, and used in the virtual screen. In other embodiments a homology model of a lipoxygenase protein may be generated and used in the virtual screen. As used herein a homology model refers to a model depicting the three-dimensional structure of a protein that is generated based on the empirically determined three-dimensional structure of a homologous protein. In some embodiments homology modeling of a lipoxygenase protein is based on the publicly available 2.4 Å resolution structure of 15-rLO. Homology modeling may also be referred to as comparative modeling or knowledge-based modeling. A nonlimiting example of a software program that could be used for homology modeling is Protein Local Optimization Program (PLOP, available from Schrodinger, Inc. within PRIME (Protein Integrated Modeling Environment)). In some embodiments the homology modeling procedure may use the OPLS all-atom force field and a Generalized Born solvent model for choosing low-energy structures.

Other nonlimiting examples of protein modeling software programs that could be used in conjunction with the instant invention, discussed further in U.S Patent Pub. No. 20070020745 include: SYBYL (Tripos Inc.); AMBER (Oxford Molecular); CERIUS2 (Molecular Simulations Inc.); INSIGHT II (Molecular Simulations); CATALYST (Molecular Simulations Inc.); QUANTA (Molecular Simulations Inc.); HYPERCHEM (Hypercube Inc.); FIRST DISCOVERY (Schrodinger Inc.), MOE (Chemical Computing Group), and CHEMSITE (Pyramid Learning).

During the virtual screening process, compounds can be tested individually for docking against a lipoxygenase protein. The term “docking” as used herein refers to aligning the three dimensional structures of molecules by computational methods, in order to identify specific binding. The virtual screen may make use of a library of compounds. In some embodiments, the ChemBridge “diversity set” will be used as a source for the compound library. Nonlimiting examples of other structural libraries, discussed further in U.S Patent Pub. No. 20070020745 include the ACD (Available Chemical Directory, MDL Inc.), AsInEx, Bionet, ComGenex, the Derwent World Drug Index (WDI), the Contact Service Company database, LaboTest, ChemBridge Express Pick, ChemStar, BioByteMasterFile, Orion, SALOR, TRIAD, ILIAD, the National Cancer Institute database (NCl), the HTS Chemicals collection (Oxford Molecular), the LeadQuest™ files (Tripos), or products from Aldrich, Fluka, Sigma, or Maybridge. The LigPrep (Schrodinger, Inc) ligand preparation software may be applied for preparing the database of small molecules for docking. In some embodiments the docking algorithm may be confirmed by testing its ability to reproduce the 15-rLO crystal structure, cocrystallized with the Roche RS7 inhibitor as a template (Gillmor, S. A. et al. (1997) Nature Struct. Biol. 4:1003-1009; Kenyon et al., 2006).

During virtual screening, each compound may be assigned a score based on its ability to dock against the three dimensional structure of the lipoxygenase protein. In some embodiments, computational docking may be performed using the GLIDE (Schrodinger, Inc) molecular docking suite, which uses a modified Chemscore algorithm, called a Glidescore, for flexible ligand docking and scores the protein ligand interactions. In certain embodiments, Glide SP “standard precision” mode may be used for the initial screen, and then hits from this screen, that are ranked according to Glidescore, may be re-screened by docking using Glide's XP “extra precision” mode. In some embodiments the MM-GBSA (molecular mechanics, Generalized Born-surface area) scoring method will also be applied to all of the protein-ligand complexes. The lists of rankings that are generated can then be used to determine which compounds should be chosen for follow-up analysis.

Further nonlimiting examples of docking algorithms that could be used in conjunction with the instant invention, discussed in U.S. Patent Pub. No. 20070020745 include DOCK (UCSF), AUTODOCK (Oxford Molecular), MOE-DOCK (Chemical Computing Group Inc.), FLExX (Tripos Inc.), GOLD (Jones et al., J. Mol. Biol. 267:727-748, 1997), AFFINITY (Molecular Simulations Inc.), C2.LigandFit (Molecular Simulations Inc.), and DOCKIT (Metaphorics LLC).

Virtual screening typically will be conducted in such a way as to identify compounds that bind to specific sites within a lipoxygenase enzyme. In some embodiments compounds are selected that bind to the active/catalytic site of the lipoxygenase protein. In other embodiments compounds are selected that bind to the allosteric site of the lipoxygenase protein. In certain embodiments compounds are selected that bind to a site other than the catalytic or allosteric sites on the lipoxygenase protein. Virtual screening can also be used to identify inhibitors that are specific for individual lipoxygenases by screening with multiple individual lipoxygenase proteins. In some embodiments of the invention, compounds identified by virtual screening that bind to a lipoxygenase protein bind to multiple lipoxygenase proteins. In other embodiments, compounds identified by virtual screening that bind to a lipoxygenase protein may have specificity for binding to a single lipoxygenase protein. In some embodiments of the invention virtual screening is conducted using homology models of 12-hLOX and/or 15-hLOX. Compounds may be identified that bind to and inhibit only 12-hLOX or only 15-hLOX, or both 12-hLOX and 15-hLOX.

According to aspects of the invention, following the virtual screen, candidate lipoxygenase inhibitor compounds are then experimentally screened for inhibition of lipoxygenase activity. In one embodiment, purified lipoxygenase proteins may be screened for inhibition by the potential inhibitor compounds by monitoring the rate of formation of the conjugated diene products at 234 nm via UV spectroscopy. Percent inhibition (% inh) may be calculated as =(1−R₁/R₀), where R, is the enzyme rate with the inhibitor present and R₀ is the control rate of the enzyme. Compounds that display potent inhibition may be screened at multiple inhibitor concentrations and fit with a standard hyperbolic equation to determine IC₅₀ values. Compounds that exhibit IC₅₀ values in the nanomolar to low micromolar range may be selected for further analysis. In some embodiments, compounds that exhibit IC₅₀ values lower than approximately 10 μM against a purified human 15-LOX-1 protein, may be selected. In some embodiments compounds may be selected that exhibit IC₅₀ values lower than 100 μM. In some embodiments compounds may be selected that exhibit IC₅₀ values lower than 1

Following confirmation that a potential inhibitor compound has the ability to inhibit a lipoxygenase protein in vitro, the compound then is selected for screening in cell-based assays to determine how the inhibitor affects lipoxygenase in a cell. For example a potential inhibitor may be screened in a cell culture-based screen in neuronal cells for neuroprotective activity. The methods described in the instant invention could incorporate the use of a variety of neuronal cell lines for these assays, including cell lines derived from human or non-human species, as would be familiar to those of skill in the art. In certain embodiments, the neuron-like cell line HT22, derived from mouse hippocampus, is used. In some embodiments glutamate is used to induce oxidative stress within the cells. Glutamate-induced oxidative stress leads to lipoxygenase-dependent cell death in HT22 cells which is prevented by inhibition of lipoxygenase. In some embodiments measuring leakage of intracellular lactic dehydrogenase (LDH) into the cell culture medium may be used as an indicator of cell death. In certain embodiments LDH is measured in medium and cell lysates using a Cytotoxicity Detection Kit (Roche). Percent cell survival after subtraction of background values may be calculated by the formula:

% survival=100×LDHcells/(LDHmedium+LDHcells).

Potential lipoxygenase inhibitors may be screened in this cell-based assay, and a potential inhibitor that is found to increase percent cell survival when used in the low micromolar concentration range may be considered to exhibit neuroprotective activity in the cell-culture based assay, and therefore may be selected as a candidate lipoxygenase inhibitor compound for further follow-up analysis.

Following confirmation that a compound has neuroprotective activity in cells, the compound then is screened for neuroprotective activity in primary neurons, e.g., from human or non-human species. In some embodiments, the primary neurons used may be from a rodent such as a mouse or rat. Similarly to the assay described for cell-culture, primary cortical neurons may be subjected to oxidative glutamate toxicity. Candidate lipoxygenase inhibitors may be screened using this assay, and an inhibitor that is found to increase percent cell survival, preferably when used in the low micromolar concentration range, is considered to exhibit neuroprotective activity in the assay.

In some embodiments a compound also is tested for neuroprotective activity in oligodendroglial cells, from human or non-human species, such as rat O4+O1− cells. O4+O1− cells may be subjected to oxidative stress by depriving them of exogenous cystine, a building block needed for synthesis of the intracellular antioxidant glutathione.

This typically leads to cell death in 24 hours. Similarly to the assays in neurons, percent cell survival may be determined by LDH measurement in medium and cell lysates of the oligodendroglial cells. Candidate lipoxygenase inhibitors may be screened in this assay, and a candidate inhibitor that is found to increase percent cell survival, preferably when used in the low micromolar concentration range, is considered to exhibit neuroprotective activity in the assay.

A compound that exhibits neuroprotective activity in primary neurons and oligondendroglial cells is one that has the potential to protect against both grey matter and white matter injury and thus may have therapeutic and diagnostic applications for brain disorders or injuries affecting areas of grey matter or white matter within the brain.

The methods of the instant invention relating to therapeutic and diagnostic applications for LOXBlock-1 and/or LOXBlock-3 are also applicable for other potential lipoxygenase inhibitors that may be isolated from further such screens.

### Neurodegenerative Disorders

Aspects of the invention relate to using LOXBlock-1 and/or LOXBlock-3 to treat a neurodegenerative disorder in a subject. As used herein, the term treat, treated, or treating when used with respect to a disorder such as a neurodegenerative disorder refers to prophylaxis or treatment of an existing disorder, including neuroprotection. A prophylactic treatment increases the resistance of a subject to development of the disease or, in other words, decreases the likelihood that the subject will develop the disease. A treatment after the subject has developed the disorder means a treatment to delay the onset of, inhibit the progression of or halt altogether the onset or progression of the particular condition (e.g., a neurodegenerative disorder). As used herein, the term “subject” refers to a human or non-human mammal. Non-human mammals include livestock animals, companion animals, laboratory animals, and non-human primates. Non-human subjects also specifically include, without limitation, chickens, horses, cows, pigs, goats, dogs, cats, rats, mice, guinea pigs, hamsters, mink, and rabbits. In some embodiments the subject is a patient. As used herein, a “patient” refers to a subject who is under the care of a physician or other health care worker, including someone who has consulted with, received advice from or received a prescription or other recommendation from a physician or other health care worker. According to aspects of the invention, a patient is typically a subject having or at risk of having a stroke or other neurodegenerative disorder.

As used herein, the term “neurodegenerative disorder” refers to disorders, diseases or conditions that are caused by the deterioration of cell and tissue components of the nervous system. Some non-limiting examples of neurodegenerative disorders include stroke, Alzheimer's disease, Parkinson's disease, Huntington's disease, Periventricular leukomalacia (PVL), amyotrophic lateral sclerosis (ALS, “Lou Gehrig's disease”), ALS-Parkinson's-Dementia complex of Guam, Friedrich's Ataxia, Wilson's disease, multiple sclerosis, cerebral palsy, progressive supranuclear palsy (Steel-Richardson syndrome), bulbar and pseudobulbar palsy, diabetic retinopathy, multi-infarct dementia, macular degeneration, Pick's disease, diffuse Lewy body disease, prion diseases such as Creutzfeldt-Jakob, Gerstmann-Straussler-Scheinker disease, Kuru and fatal familial insomnia, primary lateral sclerosis, degenerative ataxias, Machado-Joseph disease/spinocerebellar ataxia type 3 and olivopontocerebellar degenerations, spinal and spinobulbar muscular atrophy (Kennedy's disease), familial spastic paraplegia, Wohlfart-Kugelberg-Welander disease, Tay-Sach's disease, multisystem degeneration (Shy-Drager syndrome), Gilles De La Tourette's disease, familial dysautonomia (Riley-Day syndrome), Kugelberg-Welander disease, subacute sclerosing panencephalitis, Werdnig-Hoffmann disease, synucleinopathies (including multiple system atrophy), Sandhoff disease, cortical basal degeneration, spastic paraparesis, primary progressive aphasia, progressive multifocal leukoencephalopathy, striatonigral degeneration, familial spastic disease, chronic epileptic conditions associated with neurodegeneration, Binswanger's disease, and dementia (including all underlying etiologies of dementia).

In certain embodiments of the invention, LOXBlock-1 and/or LOXBlock-3 may be used to treat a subject having Alzheimer's disease, either familial or sporadic. Alzheimer's disease refers to a disorder of the brain characterized by a loss of neurons from the basal forebrain, cerebral cortex and other brain areas, leading to a loss of memory and dementia. Pathologies of Alzheimer's disease include the formation of intracellular inclusions, extracellular deposits of amyloid plaques, and neurofibrillary and granulovascular neuronal degeneration. In other embodiments of the invention, LOXBlock-1 and/or LOXBlock-3 may be used to treat a subject having Parkinson's disease. Parkinson's disease refers to a progressive disorder of the nervous system, affecting the part of the brain that controls motor activity, leading to loss of control over movement and speech.

Neurodegenerative disorders may also be the result of a brain injury or trauma including that which is caused by a stroke, an injury to the head or spinal cord, or acute ischemic injury. Ischemic injuries refer to conditions that arise when the brain receives insufficient blood flow. In some embodiments, injury to the brain or nervous system can result from a traumatic injury, or could be the result of infection, radiation, chemical or toxic damage. Injury within the brain and nervous system, which may be diffuse or localized, includes an intracranial or intravertebral lesion or hemorrhage, cerebral ischemia or infarction including embolic occlusion and thrombotic occlusion, perinatal hypoxic-ischemic injury, whiplash, shaken infant syndrome, reperfusion following acute ischemia, or cardiac arrest.

In certain embodiments, LOXBlock-1 and/or LOXBlock-3 may be used to treat a neurodegenerative disorder caused by a stroke. The term stroke, as used herein, refers to the sudden death of cells in a specific area of the brain due to disruption of blood flow to the brain. Stroke may also be referred to as cerebral accident, cerebral infarction, or cerebrovascular accident. Disruption of blood flow to the brain may be caused by blockage of an artery or an artery bursting, preventing normal blood circulation. A stroke may be preceded by transient ischemic attacks (TIAs), or a mini-stroke, which are characterized by temporarily disrupted blood flow to the brain. A stroke can lead to loss of vision, loss of speech, loss of muscular control, loss of consciousness, coma and death. In other embodiments LOXBlock-1 and/or LOXBlock-3 may be used to treat a neurodegenerative disorder caused by Periventricular leukomalacia (PVL), the most common ischemic brain injury in premature infants, involving a white matter lesion occurring in the border zone of deep penetrating arteries of the middle cerebral artery.

Neurodegenerative disorders are frequently characterized by neuronal cell death linked to oxidative stress. As used herein, “oxidative stress” refers to physiological stress caused by reactive oxygen species. Oxidative stress, which can affect a molecule, cell, organ, tissue or organism occurs when there is a disruption in the balance between production and neutralization of reactive oxygen species including free radicals and peroxides generated through the metabolism of oxygen. Oxidative stress affects multiple cellular components including DNA, protein and lipids and can lead to cell dysfunction and death. As used herein the term “oxidative stress-related injury” refers to any damage incurred by a molecule, cell, tissue, organ or organism due to oxidative stress. Aspects of the invention relate to protecting a cell against oxidative stress-related injury through the use of LOXBlock-1 and/or LOXBlock-3 to inhibit 12/15-LOX activity. In certain embodiments protection of cells against oxidative stress-related injury may occur in vitro, while in other embodiments protection of cells against oxidative stress-related injury may occur in vivo.

Aspects of the invention relate to neuroprotection. As used herein, the term “neuroprotection” refers to the prevention or reduction of damage including damage due to oxidative stress, to cells of the nervous system such as neurons and oligodendrocytes. The term “neuroprotective” refers to the capability of a compound or composition to prevent or reduce damage, including damage due to oxidative stress, of cells of the nervous system such as neurons and oligodendrocytes. In some embodiments the damage to the cells of the nervous system may be the result of a neurodegenerative disorder. In other embodiments the damage to the nervous system may be the result of an acute trauma.

### Imaging and Diagnostics

Expression of 12/15-LOX has recently been observed to increase in brain areas surrounding a stroke infarct (van Leyen et al., 2006). Thus the invention also provides methods for using LOXBlock-1, LOXBlock-3 and/or any combination thereof in detecting expression of LOX proteins, and in diagnosis of stroke or other neurodegenerative disorders.

According to aspects of the invention, a method for imaging a stroke-related infarction is provided consisting of administering to a patient an effective amount of LOXBlock-1, LOXBlock-3 and/or any combination thereof, and detecting the LOXBlock-1, LOXBlock-3 and/or any combination thereof. As discussed further below, the term “effective amount” of a compound or composition of the invention refers to the amount necessary or sufficient to realize a desired biologic effect. In some embodiments detection of LOXBlock-1, LOXBlock-3 and/or any combination thereof will be achieved through the use of neuroimaging techniques such as positron emission tomography (PET) or single photon emission computed tomography (SPECT) imaging. PET scanning refers to a method of nuclear imaging frequently used for visualizing brain function and diagnosing brain disorders, wherein a subject is provided with a radiopharmaceutical that allows visualization of chemical activity in certain regions of the brain. SPECT is a similar imaging technique to PET except that it uses radioactive substances that emit gamma rays instead of positrons and have longer half-lives than those used in PET. These and other neuroimaging techniques that may be suitable in conjunction with the instant invention are well-known to those of skill in the art.

In some embodiments LOXBlock-1, LOXBlock-3 and/or any combination thereof may be associated with a label to aid in their detection. In certain embodiments the label may be a radionuclide or a paramagnetic contrast agent. Some non-limiting examples of isotopes that may be used for labeling include ¹⁵N, ¹³C, ²H, ¹⁸F, ¹²³I, ⁷⁵Se, ³⁵S, ¹⁴C, ³H, ¹¹C, ¹⁵O, ⁷⁵Br, ¹³³Xe and ⁹⁹Tc. Further categories of labels could include but are not limited to spin labels, heavy atom labels, fluorescent, chemiluminescent or photolabile labels, digoxigenin, biotin, chelator groups, or polyvalent cations. Methods of labeling and the uses of labels for protein tracing are discussed further in US Patent Pub. No. 20070161047. Nonlimiting examples of paramagnetic contrast agents are discussed in U.S. Patent Pub. No. 20060264741. Due to the direct binding between each of LOXBlock-1 and LOXBlock-3 with 12/15-LOX, the detection of LOXBlock-1, LOXBlock-3 and/or any combination thereof indicates the presence of 12/15-LOX expression. Thus, detection of LOXBlock-1, LOXBlock-3 and/or any combination thereof could be used to monitor increased levels of 12/15-LOX expression in a patient relative to a control expression level, and thus could contribute to diagnosis of a stroke related infarct in patient.

### Administration

Aspects of the invention relate to administering therapeutic compositions. Compositions of the invention may be administered in effective amounts. An effective amount is a dosage of the composition of the invention sufficient to provide a medically desirable result. An effective amount means that amount necessary to delay the onset of, inhibit the progression of or halt altogether the onset or progression of the particular condition (e.g., a neurodegenerative disorder) being treated. An effective amount may be an amount that reduces one or more signs or symptoms of the condition (e.g., a neurodegenerative disorder). When administered to a subject, effective amounts will depend, of course, on the particular condition being treated (e.g., a neurodegenerative disorder), the severity of the condition, individual subject parameters including age, physical condition, size and weight, concurrent treatment, frequency of treatment, and the mode of administration. These factors are well known to those of ordinary skill in the art and can be addressed with no more than routine experimentation.

Actual dosage levels of active ingredients in the compositions of the invention can be varied to obtain an amount of the composition of the invention that is effective to achieve the desired therapeutic response for a particular subject, compositions, and mode of administration. The selected dosage level depends upon the activity of the particular composition, the route of administration, the severity of the condition being treated, the condition, and prior medical history of the subject being treated. However, it is within the skill of the art to start doses of the composition at levels lower than required to achieve the desired therapeutic effort and to gradually increase the dosage until the desired effect is achieved. In some embodiments, lower dosages would be required for combinations of multiple compositions than for single compositions (e.g. a composition that contains both LOX-Block1 and LOX-Block-3 may require lower dosages than a composition containing either compound singly).

The compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, infrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, or in the cornea.

Dosage forms for topical administration of a composition of this invention include powders, sprays, ointments, and inhalants as described herein. The composition is mixed under sterile conditions with a pharmaceutically acceptable carrier and any needed preservatives, buffers, or propellants which may be required.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the composition, it is desirable to slow the absorption of the composition from subcutaneous or intramuscular injection. This result can be accomplished by the use of a liquid suspension of crystalline or amorphous materials with poor water solubility. The rate of absorption of the composition then depends upon its rate of dissolution which, in turn, may depend upon crystal size and crystalline form. Alternatively, delayed absorption of a parenterally administered composition from is accomplished by dissolving or suspending the composition in an oil vehicle.

Injectable depot forms are made by forming microencapsule matrices of the composition in biodegradable polymers such a polylactide-polyglycolide. Depending upon the ratio of composition to polymer and the nature of the particular polymer employed, the rate of composition release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p 33, et seq. Liposomal encapsulation may include liposomes that are derivatized with various polymers (e.g., U.S. Pat. No. 5,013,556). In general, the formulation includes a composition of the invention and inert ingredients which protect against degradation in the stomach and which permit release of the biologically active material in the intestine.

In such solid dosage forms, the composition is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In one embodiment, the excipient or carrier increases uptake of the composition of the invention, overall stability of the composition and/or circulation time of the composition in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium stearate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The composition of the invention also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the composition of the invention, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the composition of the invention, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the composition of the invention. The composition is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl. 5): s.143-146 (1989)(endothelin-1); Hubbard et al., Annals of Internal Medicine 3:206-212 (1989) (α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a composition of the invention. Typically, each formulation is specific to the type of device employed and can involve the use of an appropriate propellant material, in addition to diluents, adjuvants, and/or carriers useful in therapy.

The composition is prepared in particulate form, preferably with an average particle size of less than 10 and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated:

Formulations suitable for use with a nebulizer, either jet or ultrasonic, typically comprise a composition of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the composition of the invention suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the composition of the invention and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the composition of the invention also is contemplated. Nasal delivery allows the passage of the composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the composition of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

### EXAMPLES

### Materials and Methods:

### Antioxidant Test

The inhibitors LOXBlock-1 (catalog number 5680672), LOXBlock-2 (6635967), and LOXBlock-3 (6640337) were obtained from ChemBridge (San Diego, Calif.) and dissolved in dimethyl sulfoxide (DMSO) at 1-20 mM concentration (1000-fold concentrated). The antioxidant activity of these compounds was assayed by monitoring the quenching of the stable free radical 1,1-diphenyl-2-picrylhydrazyl (DPPH) upon reaction with the testing compound (Wang et al. 2004). The decrease in optical absorbance at 517 nm was monitored using a spectrophotometer (Lambda 40, Perkin Elmer). The rate of reaction is proportional to the antioxidant potency of the test compounds. A known free radical scavenger, nordihydroguaiaretic acid (NDGA), was used as a positive control. 10 μL, of 1 mM testing reagents to achieve final concentrations of 5 μM were added to 2 mL of 500 μM DPPH stirring in a cuvette. Optical absorbance was monitored and recorded at 25 second intervals as described (Wang et al. 2004).

### HT22 Cell Culture

Maintenance and incubation of HT22 cells was carried out as described (van Leyen et al. 2005). Briefly, HT22 cells were cultured in DMEM containing 10% fetal bovine serum and penicillin/streptomycin (all media from Invitrogen). For viability experiments, cells were seeded at 5×10⁵ cells/well in 24-well plates (Corning) and treated when approximately 70% confluent. Treatment consisted of exchanging the medium with fresh medium containing the inhibitor or DMSO (0.1% final concentration), then adding 5 mM glutamate 5 minutes later. After 24 h incubation in the presence or absence of inhibitor, lactate dehydrogenase (LDH) was measured in the medium and in cell lysates from each well using a Cytotoxicity Detection Kit (Roche). Percent survival was calculated after subtraction of background values by the formula % survival=100×LDHcells/(LDHmedium+LDHcells). Data is presented as mean±SEM, averaged from three separate experiments performed in duplicate. Statistical significance was determined using the Tukey-Kramer HSD test. The results from these tests are generally similar to the outcome detected with the MTT assay in this model.

### Rat Primary Cortical Neurons

Primary Neurons were isolated from E17 rats and treated as described (Ratan et al. 2002; van Leyen et al. 2005). This protocol typically results in a culture enriched to over 90% in neuronal cells at 18 hr after seeding. To induce oxidative stress, the medium was exchanged against fresh DMEM/10% fetal bovine serum (FBS). Inhibitors or DMSO (final concentration 0.1%) were added, followed after 5 min by glutamate (final concentration 5 mM) as indicated. After 24 hr of incubation, cells were lysed by removal of medium and addition of 0.5% Triton X-100. Because of the high background values in the medium, % survival was calculated from the intracellular LDH values compared to control-treated cells. Data is presented as mean±SEM, averaged from three separate experiments performed in duplicate. Statistical significance was determined using the Tukey-Kramer HSD test.

### Rat Primary Oligodendroglial Cells

Primary oligodendroglial cells (O4+O1−); preoligodendrocytes) were isolated, maintained and treated as described (Wang et al. 2004). For treatment, the cells were washed two times with medium containing basic fibroblast growth factor and platelet derived growth factor (both from Peprotech, Princeton, N.J.), but lacking cystine. After these washing steps, the cells were incubated in fresh medium lacking cystine for 24 h in the presence or absence of inhibitors. Percent survival was determined by LDH measurement in medium and cell lysates. Data is presented as mean+/−SEM, averaged from three separate experiments performed in duplicate. Statistical significance was determined using the Tukey-Kramer HSD test.

### Results and Discussion:

Novel Lipoxygenase Inhibitors LOXBlock-1, -2, and -3, have Low Antioxidant Activities

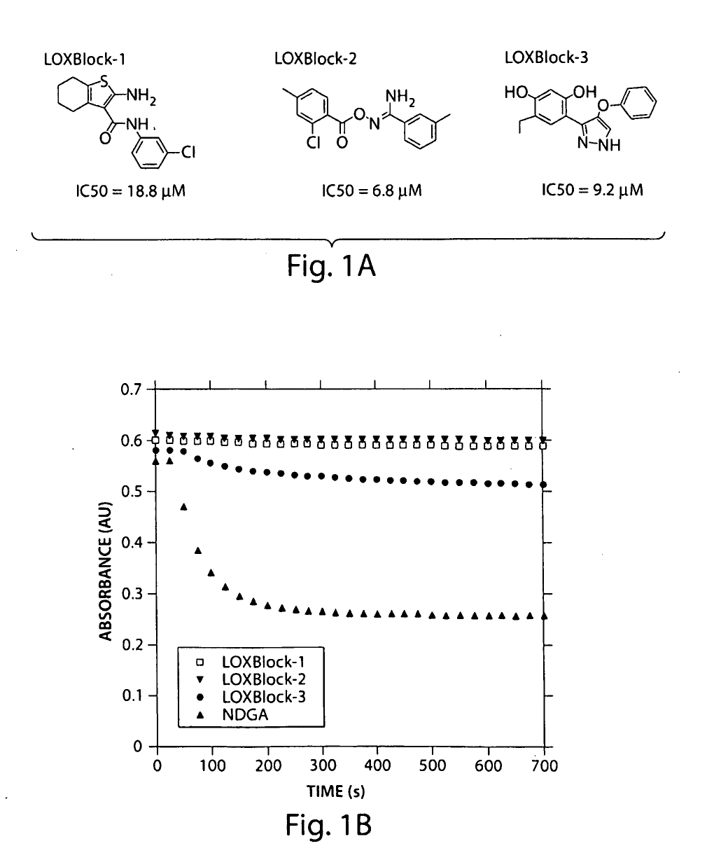

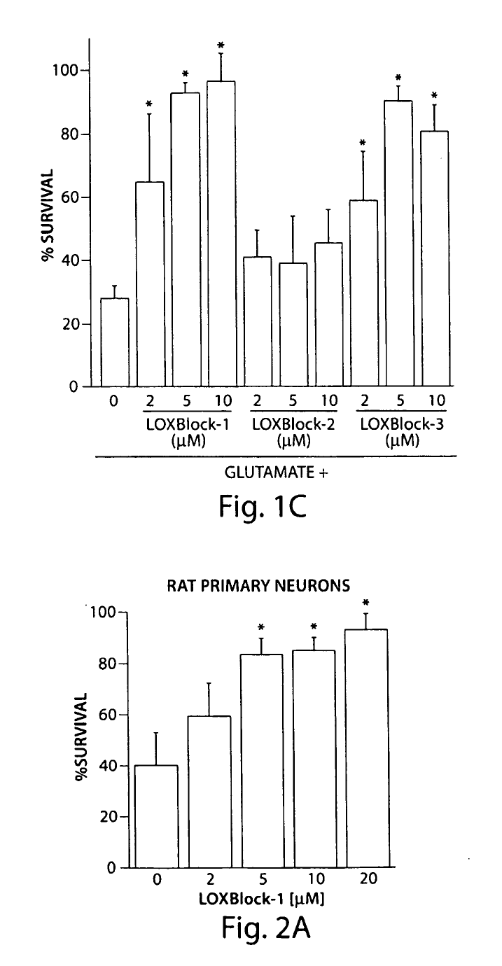

In preliminary experiments described elsewhere, a virtual screen of a library of 50,000 drug-like compounds was performed by computer modeling against the homology models of human 15-LOX-1 and human 12-LOX (Kenyon et al. 2006). The 20 hits derived from this screen were then tested in an enzymatic assay against the recombinant human 15-LOX-1, leading to three candidates with IC₅₀s in the low micromolar range. The three inhibitors have been termed LOXBlock-1, LOXBlock-2, to and LOXBlock-3 (FIG. 1A). The structures of the compounds are clearly not related to one another, reinforcing the utility of the virtual screening/in vitro testing approach.

To demonstrate that general redox chemistry was not the mechanism of inhibition, the antioxidant activity of these compounds was compared, because many antioxidants are also known to inhibit lipoxygenase. Nordihydroguaiaretic acid (NDGA), here used as positive control, is known as both a LOX inhibitor and a strong antioxidant (Whitman et al. 2002). Correspondingly, it led to rapid quenching of a stable radical, DPPH, in a well established antioxidant assay (Wang et al. 2004). In contrast, the three compounds studied showed either no (LOXBlock-1 and LOXBlock-2) or little (LOXBlock-3) antioxidant activity in this assay (FIG. 1B).

### LOXBlock-1 and -3 Protect Against Oxidative Glutamate Toxicity

Next, these compounds were tested in a simple cell culture-based screen, in which exogenously added glutamate leads to cell death through oxidative stress in a neuron-like mouse hippocampal cell line, HT22. This form of oxidative stress has previously been shown to depend on glutathione depletion and activation of 12/15-LOX (Li et al. 1997). Conversely, glutamate receptors do not appear to be responsible for the toxicity of glutamate in this model. Measuring leakage of intracellular LDH into the cell culture medium as an indicator of cell death, the compounds LOXBlock-1 and LOXBlock-3 showed strong neuroprotection at low micromolar concentration (FIG. 1C). In contrast, the third compound, LOXBlock-2, did not show significant benefit. In separate experiments carried out in the absence of glutamate (data not shown), LOXBlock-2 showed some toxicity against these cells, suggesting that LOXBlock-2 may also impact other unknown targets besides 12/15LOX.

### Rat Primary Cortical Neurons are Protected Against Oxidative Stress by LOXBlock-1 and -3

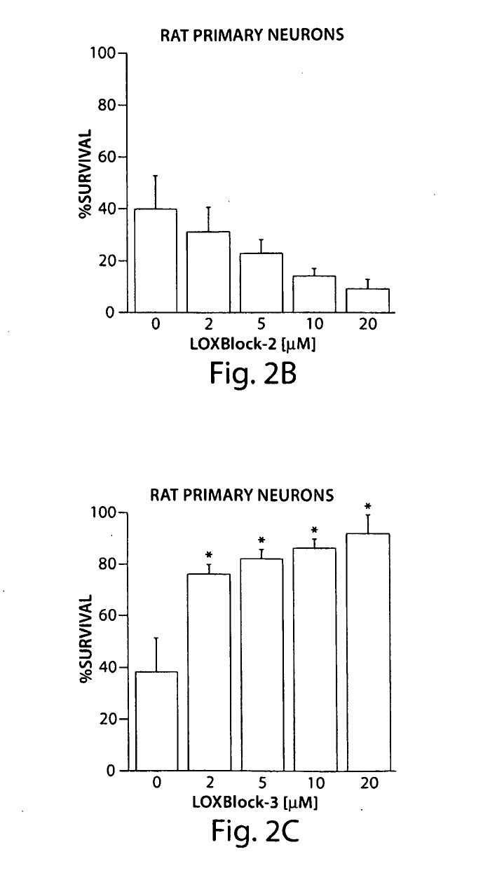

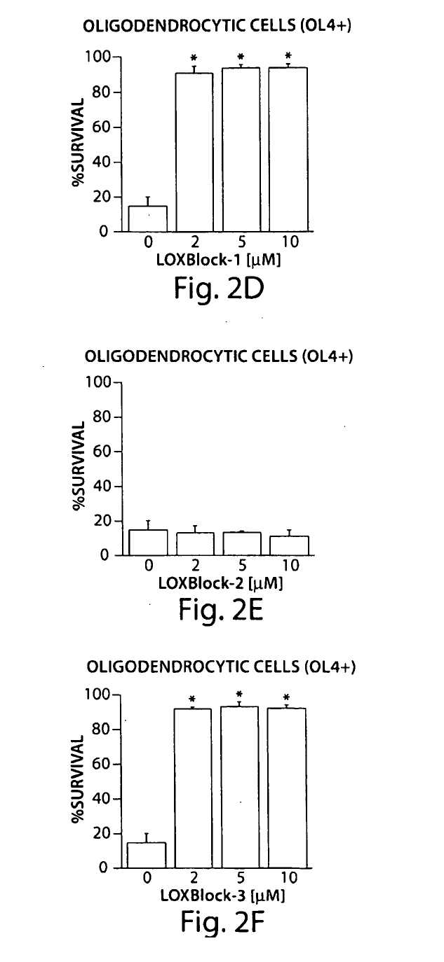

While neuronal cell lines like HT22 are excellent screening tools and very well suited for mechanistic studies, they do not always accurately reflect all characteristics of the primary cells they originate from. To further investigate whether the protection through the novel LOX inhibitors also applies to brain-derived cells in primary culture, rat cortical primary neurons were subjected to oxidative glutamate toxicity. Increasing amounts of LOXBlock-1 and of LOXBlock-3 protected primary neurons in a dose-dependent manner (FIGS. 2A and C, respectively). Again, LOXBlock-2 did not provide protection against glutamate treatment, and increasing concentrations of LOXBlock-2 led to increased cell death (FIG. 2B).

### LOXBlock-1 and -3 Protect Oligodendroglial Cells

In addition to injury to neurons in the brain, recent studies have highlighted the importance of white matter deficiencies for neurological damage. It was therefore investigated whether the novel LOX inhibitors might also protect oligodendrocytes against oxidative stress. Primary oligodendroglial cells (O4+O1−) are subjected to oxidative stress when deprived of exogenous cystine, a building block needed for synthesis of the intracellular antioxidant glutathione. Similar to oxidative glutamate toxicity but in the absence of added glutamate, a depletion of intracellular glutathione follows. This typically leads to cell death after 24 hours. In a strikingly similar result to what was seen in neurons, both LOXBlock1 and LOXBlock-3 were efficient protectors against oxidative stress (FIG. 2D-F). Interestingly, almost full protection was reached at 2 μM concentration for each compound, indicating that oligodendroglial cells are protected even more efficiently than primary neurons. Taken together, these results suggest that the novel LOX inhibitors LOXBlock-1 and LOXBlock-3 have the potential to protect against both gray matter and white matter injury.

Reported herein is an efficient screening process for evaluating novel lipoxygenase inhibitors for their neuroprotective qualities. The path chosen to identify these candidate lipoxygenase inhibitors provides several advantages over more traditional methods. Since these compounds have been tested against the human enzyme, any positive results in animal models are likely to translate well to the human situation. With a relatively simple four-step process, two novel candidates have been filtered out of a library of 50,000 druglike compounds that had previously not been evaluated for their neuroprotective qualities. The initial steps, identifying molecules that could bind the active site of human 15 lipoxygenase in a computer-based screen, followed by testing the in vitro efficacy in inhibiting the enzymatic activity of recombinant human 15-LOX, have been described in a previous publication (Kenyon et al. 2006). To this was added a simple cellular assay to measure protection against oxidative stress in a neuronal cell line, HT22. These cells are easy to handle and could readily be adapted for high-throughput screening. Glutamate-induced oxidative stress leads to a lipoxygenase-dependent cell death in this cell line (Li et al. 1997). Of the three compounds that had been shown to inhibit human 15-LOX in vitro, two efficiently protected the HT22 cells against oxidative glutamate toxicity, LOXBlock-1 and LOXBlock-3. The third, labeled LOXBlock-2, was not an effective neuroprotectant in this assay.

Because of this surprising result, the experiment was repeated with both higher and lower concentrations of LOXBlock-2, ranging from 100 nM to 40 μM, to investigate whether protection could be achieved at these concentrations. Solubility in DMSO of LOXBlock-2 appears to be satisfactory, in that higher concentrations of the stock solution (40 mM) appeared clear, without a residual pellet. The lower concentrations were at best marginally protective against glutamate, but 20 μM and 40 LOXBlock-2 led to reduced survival of HT22 cells, suggesting some toxicity of the compound.

Following this screening process, rat-derived primary brain cells were tested for protection against this form of lipoxygenase-mediated cell death. It has recently become clear that, to protect against ischemia/reperfusion injury, it is not sufficient to salvage gray matter, but white matter must also be rescued (Dewar et al. 1999). It is therefore useful to investigate the effect of a neuroprotective drug not just on neurons, but also on the oligodendrocytes that provide the supportive myelin sheaths for neuronal axons. Both primary neurons and cells of the oligodendrocyte lineage were protected with similar efficiency by LOXBlock-1 and by LOXBlock-3, whereas LOXBlock-2 again provided no protection. Possibly, LOXBlock-2 also affects another target besides 12/15-LOX, but in any case this compound does not seem to be a good candidate for further studies.

Surprisingly, both LOXBlock-1 and LOXBlock-3 were protective at lower concentrations than their inhibitory concentrations against the human 15-LOX in vitro. Several factors may account for this discrepancy. The efficacy against mouse and rat 12/15-LOX may be somewhat higher than that against the human enzyme. Also, the experimental conditions used to measure the inhibition of human 15-LOX in vitro may not be optimal; indeed, the addition of detergent can have a major impact on the inhibitory concentration measured. In this context, it is interesting to note that the lipoxygenase-dependent cell death in neural cells may depend on the oxidation of intracellular membranes, not of free arachidonic acid, as measured in the in vitro screen. In any case, the fairly good protection afforded by both LOXBlock-1 and LOXBlock-3 even at 2 μM concentration suggests that these may be efficient neuroprotectants. Importantly, these compounds were identified in a computer screen by their potential to fit into the active site of human 12-LOX or human 15-LOX. This, together with the finding that LOXBlock-3 exhibits only mildly antioxidant properties and LOXBlock-1 is clearly not an antioxidant, suggests that these inhibitors function through direct inhibition of 12/15-LOX.

The results of this study may be applicable to neuroprotection in a variety of neurodegenerative diseases where oxidative stress is a major cause of injury. The best-studied example of this may be stroke, one of the deadliest and most debilitating diseases. Much has been learned in recent years about mechanisms leading to brain damage following stroke, but as yet this knowledge has not translated into successful drug compounds. At the same time, there is a dire need for treatment options, given that tissue plasminogen activator (tPA) is currently the only drug with FDA approval. Similarly, although there are therapies that relieve symptoms in Alzheimer's and Parkinson's disease, no effective neuroprotectants exist for these chronic neurodegenerative disorders either. Furthermore, LOX inhibitors might be suitable as preventive agents in premature infants and sick term infants, whose developing brains seem to be very susceptible to oxidative injury. Oxidative stress to preoligodendrocytes in the developing white matter has been implicated in the pathogenesis of periventricular leukomalacia, the lesion underlying most cases of cerebral palsy in premature infants (Gerstner et al. 2006; Wang et al. 2004). Therefore, the LOX inhibitors identified by our step-wise screening process here may provide new opportunities for targeting a wide range of disorders. The two novel 12/15-LOX inhibitors, LOXBlock-1 and LOXBlock-3, may now be evaluated for their pharmacological properties in vivo.

### REFERENCES

Brash A R. (1999). Lipoxygenases: occurrence, functions, catalysis, and acquisition of substrate. J Biol Chem 274(34):23679-23682.
Dewar D, Yam P, McCulloch J. (1999). Drug development for stroke: importance of protecting cerebral white matter. Eur J Pharmacol 375(1-3):41-50.
Gerstner B, Buhrer C, Rheinlander C, Polley O, Schuller A, Bems M, Obladen M, Felderhoff-Mueser U. (2006). Maturation-dependent oligodendrocyte apoptosis caused by hyperoxia. J Neurosci Res 84(2):306-315.
Jacobson M P, SalI A. (2004). Comparative Protein Structure Modeling and Its Applications to Drug Discovery. Annu Rep Med Chem 39:259-276.
Kenyon V, Chomy I, Carvajal W J, Holman T R, Jacobson M P. (2006). Novel human lipoxygenase inhibitors discovered using virtual screening with homology models. J Med Chem 49(4):1356-1363.
Khanna S, Roy S, Slivka A, Craft T K, Chaki S, Rink C, Notestine M A, DeVries A C, Parinandi N L, Sen C K. (2005). Neuroprotective properties of the natural vitamin E alpha-tocotrienol. Stroke 36(10):2258-2264.
Kuhn H, Belkner J, Wiesner R, Brash A R. (1990). Oxygenation of biological membranes by the pure reticulocyte lipoxygenase. J Biol Chem 265(30):18351-18361.
Lebeau A, Terro F, Rostene W, Pelaprat D. (2004). Blockade of 12-lipoxygenaseexpression protects cortical neurons from apoptosis induced by beta-amyloid peptide. Cell Death Differ 11(8):875-884.
Li Y, Maher P, Schubert D. (1997). A role for 12-lipoxygenase in nerve cell death caused by glutathione depletion. Neuron 19(2):453-463.
Lin M T, Beal M F. (2006). Mitochondrial dysfunction and oxidative stress in neurodegenerative diseases. Nature 443(7113):787-795.
Mytilineou C, Kramer B C, Yabut J A. (2002). Glutathione depletion and oxidative stress. Parkinsonism Relat Disord 8(6):385-387.
Pratico D, Zhukareva V, Yao Y, Uryu K, Funk C D, Lawson J A, Trojanowski J Q, Lee V M. (2004). 12/15-lipoxygenase is increased in Alzheimer's disease: possible involvement in brain oxidative stress. Am J Pathol 164(5):1655-1662.
Ratan R R, Ryu H, Lee J, Mwidau A, Neve R L. (2002). In vitro model of oxidative stress in cortical neurons. Methods Enzymol 352:183-190.
van Leyen K, Duvoisin R M, Engelhardt H, Wiedmann M. (1998). A function for lipoxygenase in programmed organelle degradation. Nature 395(6700):392-395.
van Leyen K, Kim H Y, Lee S R, Jin G, Arai K, Lo E H. (2006). Baicalein and 12/15 lipoxygenase in the ischemic brain. Stroke 37(12):3014-3018.
van Leyen K, Siddiq A, Ratan R R, Lo E H. (2005). Proteasome inhibition protects HT22 neuronal cells from oxidative glutamate toxicity. J Neurochem 92(4):824-830.
Wang H, Li J, Follett P L, Zhang Y, Cotanche D A, Jensen F E, Volpe J J, Rosenberg P A. (2004). 12-Lipoxygenase plays a key role in cell death caused by glutathione depletion and arachidonic acid in rat oligodendrocytes. Eur J Neurosci 20(8):2049-2058.
Whitman S, Gezginci M, Timmermann B N, Holman T R. (2002). Structure-activity relationship studies of nordihydroguaiaretic acid inhibitors toward soybean, 12 human, and 15-human lipoxygenase. J Med Chem 45(12):2659-2661.
Yamamoto S, Suzuki H, Nakamura M, Ishimura K. (1999). Arachidonate 12-lipoxygenase isozymes. Adv Exp Med Biol 447:37-44.
Zhang Y, Wang H, Li J, Jimenez D A, Levitan E S, Aizenman E, Rosenberg P A. (2004). Peroxynitrite-induced neuronal apoptosis is mediated by intracellular zinc release and 12-lipoxygenase activation. J Neurosci 24(47):10616-10627.

### EQUIVALENTS

It should be understood that the preceding is merely a detailed description of certain embodiments. It therefore should be apparent to those of ordinary skill in the art that various modifications and equivalents can be made without departing from the spirit and scope of the invention, and with no more than routine experimentation.

All references, patents and patent applications that are recited in this application are incorporated by reference herein in their entirety.

What is claimed is:

1. A method for treating a neurodegenerative disorder in a subject, the method comprising:
administering to a subject having or suspected of having a neurodegenerative disorder a pharmaceutical composition comprising LOXBlock-1, LOXBlock-3 and/or any combination thereof in an amount effective to treat the neurodegenerative disorder.

2. The method of claim 1 wherein the neurodegenerative disorder is stroke.

3. The method of claim 1 wherein the neurodegenerative disorder is Alzheimer's disease.

4. The method of claim 1 wherein the neurodegenerative disorder is Parkinson's disease.

5. The method of claim 1 wherein the neurodegenerative disorder is periventricular leukomalacia (PVL).

6. The method of claim 1 wherein the subject is a human.

7. The method of claim 1 wherein the pharmaceutical composition further comprises at least one pharmaceutically acceptable carrier, diluent, excipient or adjuvant.

8. The method of claim 1 wherein the method of administration is parenteral, oral, buccal, pulmonary, intravenous, intramuscular, subcutaneous, aural, rectal, vaginal, ophthalmic, intradermal, intraoccular, intracerebral, intralymphatic, intraarticular, intrathecal or intraperitoneal.

9. A method for protecting a cell against oxidative-stress-related injury by inhibition of 12/15-LOX activity, the method comprising:
contacting a cell undergoing oxidative-stress-related injury with LOXBlock-1, LOX-Block-3 and/or any combination thereof in an amount effective to inhibit 12/15-LOX activity.

10. The method of claim 9 wherein the cell is neuronal.

11. The method of claim 9 wherein the cell is oligodendroglial.

12. The method of claim 9 wherein the cell is in vitro.

13. The method of claim 9 wherein the cell is in vivo.

14. The method of claim 9 wherein the cell is a human cell.

15. The method of claim 9 wherein the cell is a non-human cell.

16. The method of claim 15 wherein the cell is a rodent cell.

17. The method of claim 16 wherein the cell is an HT22 cell.

18. The method of claim 9 wherein the cell is a plurality of cells that comprise more than one cell type.

19. The method of claim 13 wherein the oxidative-stress-related injury comprises a neurodegenerative disorder.

20. The method of claim 19 wherein the neurodegenerative disorder is stroke.

21-38. (canceled)
